(19) United States
(12) Patent Application Publication
(10) Pub. No.: US 2025/0180550 A1
(43) Pub. Date: Jun. 5, 2025

(54) SOLID-PHASE CARRIER AND KIT FOR MEASURING AN ANALYTE

(71) Applicants: TOKUSHIMA UNIVERSITY, Tokushima-Shi (JP); SYSMEX CORPORATION, Kobe-Shi (JP)

(72) Inventors: Masayuki Ono, Yokohama-Shi (JP); Yoshiya Miyasaka, Yokohama-Shi (JP); Takaaki Yano, Tokushima-Shi (JP); Masaya Okada, Kobe-Shi (JP)

(73) Assignees: TOKUSHIMA UNIVERSITY, Tokushima-Shi (JP); SYSMEX CORPORATION, Kobe-Shi (JP)

(21) Appl. No.: 19/045,067

(22) Filed: Feb. 4, 2025

Related U.S. Application Data
(63) Continuation of application No. PCT/JP2023/028745, filed on Aug. 7, 2023.

(30) Foreign Application Priority Data
Aug. 8, 2022 (JP) 2022-126678

(51) Int. Cl. G01N 33/543; G01N 21/552; G01N 33/573

(52) U.S. Cl. CPC G01N 33/54373; G01N 21/554; G01N 33/54393; G01N 33/573; G01N 2333/96433

(57) ABSTRACT

Disclosed is a solid-phase carrier used for measuring an analyte, comprising: a substrate; and a nanostructured material disposed on one surface of the substrate, wherein the nanostructured material comprises: a plasmon excitation layer formed of a material capable of exciting surface plasmon resonance and having a nanostructure in which localized surface plasmon resonance is excited by irradiation of light; and a coating layer formed of a material that does not excite surface plasmon resonance, wherein the plasmon excitation layer includes a coated region covered by the coating layer and an uncoated region not covered by the coating layer, wherein the uncoated region includes a hotspot where localized surface plasmon resonance occurs, and wherein a surface of the coating layer has a lower binding affinity for a specific binding substance of the analyte than a surface of the plasmon excitation layer.

### CROSS REFERENCE TO RELATED APPLICATIONS

This application is a continuation of International Application PCT/JP 2023/028745 filed on Aug. 7, 2023, which claims benefit of Japanese patent application JP 2022-126678 filed on Aug. 8, 2022, both of which are incorporated herein by reference in their entireties.

### FIELD OF THE INVENTION

The present invention relates to a solid-phase carrier and a kit for measuring an analyte.

### BACKGROUND OF THE INVENTION

Measurement techniques using surface plasmon resonance (SPR) phenomena have been widely used for analysis of biological samples or analysis of intermolecular interactions. As a representative commercially available devices, Biacore and the like can be mentioned. In these apparatuses, a measurement substrate in which a gold thin film of about 50 nm is formed on a glass surface is used. The lower detection limit concentration of biomolecules such as proteins by these apparatuses is several hundred pM to several nM.

Techniques using nanophotonics have been studied as a technique that enables more sensitive measurement using plasmon phenomena. In nanophotonics, by using a nanoscale structure, a specific optical phenomenon is generated, and detection performance of a sensor can be improved by an effect of the optical phenomenon. For example, measurement techniques using localized plasmon resonance (LSPR) phenomena are beginning to be used. The LSPR is a phenomenon in which a specific energy (wavelength) is enhanced in the vicinity of a nanostructure by confinement of light to the nanostructure, and the confinement energy varies depending on shape and material of the nanostructure.

These nanostructures can be formed on a surface of a substrate using microfabrication techniques such as electron beam lithography and laser lithography. Nanostructures may also be fabricated on a surface of a substrate using methods such as coating nanoparticles.

A method for fabricating a nanohole structure for the LSPR using the microfabrication techniques is described in, for example, Japanese Laid-Open Patent Publication No. 2009-222401A. As nanostructures, pillar-shaped or sword-shaped nanostructures may also be formed. In some cases, a mold is formed by the microfabrication techniques, a nanostructure is formed on a surface of a substrate by molding techniques such as nanoimprinting, and then a substance having plasmon characteristics such as gold or silver is formed into film by methods such as vacuum film formation, thereby a nanostructure for the LSPR is formed. There is also a method of fabricating a nanostructure on a surface of aluminum or the like by anodization methods.

In a measurement technique using the LSPR phenomenon, an analyte is trapped or adsorbed on a surface of a nanostructure. This results in a spectral shift of a resonant wavelength. By detecting this spectral shift, the analyte can be quantified. The spectrum of the resonance wavelength of the LSPR can be identified, for example, by detecting transmitted light transmitted through the nanostructure.

The LSPR occurs only in a specific part of the nanostructure. This portion is called a hotspot. For example, in a case of a nanohole shape, an edge portion of the hole is the hotspot. In a case of s sword shape, a top portion of the sword is the hot spot.

When an analyte is measured using the LSPR, spectral shift does not occur if the analyte is captured in a region other than the hotspot. Rather, the analyte captured in the hotspot is reduced because the analyte is captured in a region other than the hotspot. As a result, the spectral shift amount is reduced (S/N is deteriorated). In some cases, when concentration of the analyte is low and the analyte is captured in a region other than the hotspot, the spectral shift amount becomes small, and thus quantification of the analyte cannot be accurately performed.

### SUMMARY OF THE INVENTION

The scope of the present invention is defined solely by the appended claims, and is not affected to any degree by the statements within this summary.

A solid-phase carrier used for measuring an analyte comprises a substrate; and a nanostructured material disposed on one surface of the substrate, wherein the nanostructured material comprises: a plasmon excitation layer formed of a material capable of exciting surface plasmon resonance and having a nanostructure in which localized surface plasmon resonance is excited by irradiation of light; and a coating layer formed of a material that does not excite surface plasmon resonance, wherein the plasmon excitation layer includes a coated region covered by the coating layer and an uncoated region not covered by the coating layer, wherein the uncoated region includes a hotspot where localized surface plasmon resonance occurs, and

wherein a surface of the coating layer has a lower binding affinity for a specific binding substance of the analyte than a surface of the plasmon excitation layer.

### BRIEF DESCRIPTION OF THE DRAWINGS

FIG. 1A shows an example of a nanohole structure.

FIG. 1B shows simulation results of LSPR characteristics generated in the nanohole structure of FIG. 1A using a theoretical calculation method based on electromagnetic wave analysis.

FIG. 2A is a schematic diagram showing an example of a solid-phase carrier. FIG. 2A shows a perspective view.

FIG. 2B shoes a cross-sectional view of the solid-phase carrier of FIG. 2A taken along line A-A.

FIG. 3 is a schematic diagram illustrating an example of a method for producing the solid-phase carrier.

FIG. 4 is a schematic diagram showing an example of the solid-phase carrier to which a first specific binding substance is bound.

FIG. 5 is a schematic diagram illustrating an example of a method of measuring a analyte.

FIG. 6A is a schematic diagram illustrating an example of a method for detecting an LSPR phenomenon.

FIG. 6B shows an example of a peak shift of a transmitted light spectrum due to binding of the analyte.

FIG. 7 shows an example in which a change in transmittance at a specific wavelength is measured in real time when a first specific binding substance (antibody) is reacted with the solid-phase carrier 1, and a blocking step and an analyte capturing step are performed.

FIG. 8 is a schematic diagram illustrating an example of a method of measuring the analyte using a labeling substance.

FIG. 9A is a schematic diagram illustrating an example of a method for detecting an LSPR phenomenon.

FIG. 9B shows an example of a peak shift of a transmitted light spectrum due to binding of the analyte and the labeling substance.

FIG. 10 shows an example in which a change in transmittance at a specific wavelength is measured in real time when a first specific binding substance (antibody) is reacted with the solid-phase carrier 1, a blocking step and an analyte capturing step are performed.

FIG. 11A is a schematic diagram showing solid-phase carriers of an example and a comparative example after the analyte capturing step.

FIG. 11B is a graph plotting amounts of change in transmittance when PSA of 0 to 1 ng/ml is reacted with the solid-phase carrier of the example or the comparative example.

### DETAILED DESCRIPTION

Hereinafter, embodiments of the present invention will be described in detail with reference to the drawings. In the drawings, the same or corresponding parts are denoted by the same or corresponding reference numerals, and redundant description thereof will be omitted. The dimensional ratios in the drawings may be exaggerated for the purpose of explanation, and do not necessarily coincide with actual dimensional ratios.

The term “comprise” means that a component other than the component of interest may be included. The term “consisting of” means that components other than those of interest are excluded. The term “consist essentially of” means that components other than those of interest are not included in embodiments that perform a specific function (e.g., an embodiment that completely loses effectiveness of invention). In the present specification, the term “comprise” encompasses embodiments using “consist of” and embodiments using “consist essentially of”.

Proteins, antibodies, peptides, nucleic acids (DNA, RNA), extracellular vesicles (such as exosomes), sugar chains, lectins, and lipids can be isolated. “Isolated” means a natural state or a state separated from other components. “Isolated” component may be substantially free of other components. “Substantially free of other components” means that an amount of the other components contained in the isolated component is negligible. The amount of the other components contained in the isolated component may be, for example, 10% by mass or less, 5% by mass or less, 4% by mass or less, 3% by mass or less, 2% by mass or less, 1% by mass or less, 0.5% by mass or less, or 0.1% by mass or less. The proteins, antibodies, peptides, nucleic acids (DNA, RNA), extracellular vesicles (such as exosomes), sugar chains, lectins, and lipids described herein can be isolated proteins, isolated antibodies, isolated peptides, isolated nucleic acids (isolated DNA, isolated RNA), isolated extracellular vesicles (such as exosomes), isolated sugar chains, isolated lectins, and isolated lipids, respectively.

“Analyte” means a substance to be measured (measurement substance). The analyte is not particularly limited, and any substance may be the analyte as long as a specific binding substance that specifically binds to the analyte is present. The specific binding substance may specifically bind to a molecule possessed by the analyte. Examples of the analyte include, but are not limited to, exosomes, peptides, proteins, nucleic acids, sugar chains, lectins, lipids, and the like.

The analyte may be included in, for example, a biological sample. The biological sample is not particularly limited, and a sample collected from a living body can be used. Biological samples include, but are not limited to, body fluid samples such as blood, serum, plasma, saliva, urine, tears, sweat, milk, nasal discharge, semen, pleural effusion, gastrointestinal secretions, cerebrospinal fluid, interstitial fluid, and lymphatic fluid, cell debris samples, cell extract samples, and the like. The biological sample may be obtained by extracting or concentrating a specific biological substance fraction.

“Specific binding substance” means a substance that specifically binds to a specific molecule. “Specifically binds” means having a high binding affinity for a specific molecule, but having a very low binding affinity for other molecules. The specific binding substance preferably has high binding to certain molecules, but little binding to other molecules. In one embodiment, the particular molecule is a biomolecule. Biomolecules include, but are not limited to, proteins, peptides, nucleic acids, sugar chains, and the like.

Combinations of a biomolecule and a specific binding substance include, but is not limited to, for example:
a peptide or protein, and an antibody;
a nucleic acid, and a nucleic acid comprising a sequence complementary to a sequence contained in the nucleic acid;
a ligand, and a receptor thereof;
an enzyme, and its substrate, inhibitor, or cofactor;
a sugar chain, and a lectin;
a peptide, a protein, a nucleic acid, or the like, and an aptamer;
a transcriptional control sequence portion of a nucleic acid, and its transcriptional control factor; and the like.

“A specific binding substance of a target substance” means a substance that specifically binds to the analyte. When the analyte is a peptide or a protein, the specific binding substance of the analyte includes, for example, an antibody or an aptamer, and an antibody is preferable.

“Antibody” means an immunoglobulin having antigen-binding activity. The antibody need not be an intact antibody, as long as it has antigen-binding activity, and may be an antigen-binding fragment. As used herein, the term “antibody” encompasses antigen-binding fragments. “Antigen-binding fragment” is a polypeptide comprising a portion of an antibody and maintaining the antigen-binding activity of the original antibody. Preferably, the antigen-binding fragments all comprise six complementarity-determining regions (CDR) of the original antibody. That is, it is preferable to include all of CDR1, CDR2, and CDR3 of the heavy chain variable region and CDR1, CDR2, and CDR3 of the light chain variable region. Examples of the antigen-binding fragment include Fab, Fab′, F(ab′)₂, a variable-region fragment (Fv), a disulfide-bonded Fv, a single-chain Fv (scFv), and a sc (Fv)₂.

The antibody may be derived from any organism. Examples of the organism from which the antibody is derived include, but are not limited to, mammals (human, mouse, rat, rabbit, horse, cow, pig, monkey, dog, etc.), birds (chicken, ostrich), and the like.

Antibodies may be of any class and subclass of immunoglobulins. The antibody may be a monoclonal antibody or a polyclonal antibody, but a monoclonal antibody is preferred.

The antibody can be produced by a known method such as an immunization method, a hybridoma method, or a phage display method.

### [Solid-Phase Carrier]

A first aspect of the present invention is a solid-phase carrier. The solid-phase carrier includes a substrate and a nanostructured material disposed on one surface of the substrate. The nanostructured material includes a plasmon excitation layer formed of a material capable of exciting surface plasmon resonance and having a nanostructure in which localized surface plasmon resonance is excited by irradiation of light, and a coating layer formed of a material that does not excite surface plasmon resonance. The plasmon excitation layer includes a coated region covered by the coating layer and an uncoated region not covered by the coating layer, and the uncoated region includes a hotspot where localized surface plasmon resonance is enhanced. The surface of the coating layer has a lower binding affinity for a specific binding substance of the analyte than the surface of the plasmon excitation layer.

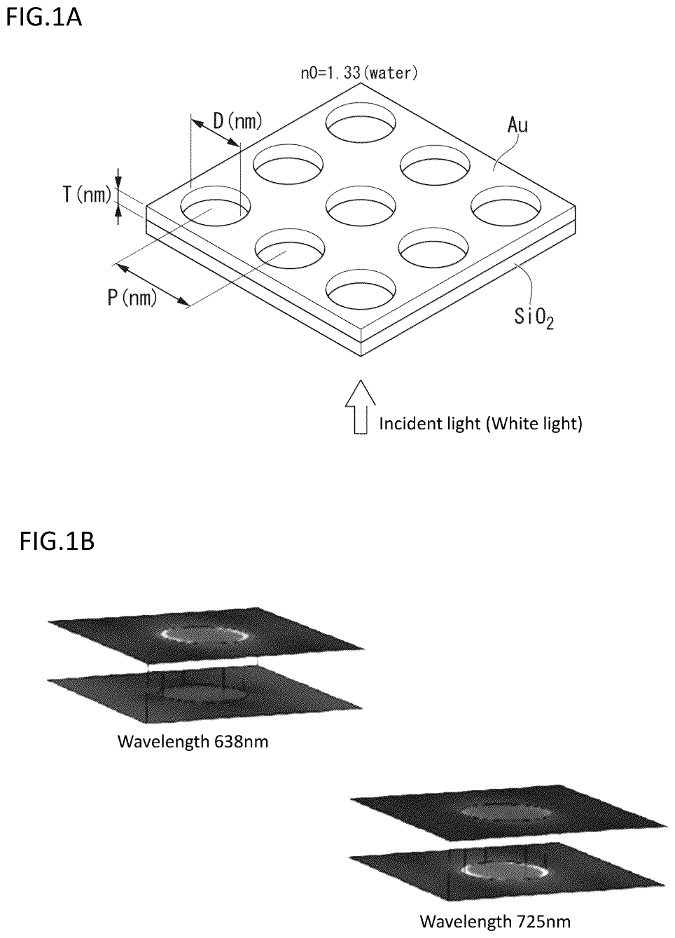

The nanostructure in which the LSPR phenomenon occurs includes a nanohole structure. FIG. 1A shows an example of a nanohole structure. FIG. 1B shows the results of simulating the LSPR characteristics of the nanohole structure of FIG. 1A using a theoretical calculation method using electromagnetic wave analysis. Periodically arrayed gold nanohole structure on a glass (SiO₂) substrate is irradiated with white light from the glass substrate side, and transmitted light spectrum, while transmitted light being detected from the gold nanohole side, were calculated by optical simulations (COMSOL Multiphysics) using the finite element method. The pitch (P) of the nanoholes was 400 nm, the diameter (D) of the nanoholes was 150 nm, and the depth (T) of the gold nanoholes was 100 nm. In FIG. 1A, n0=1.33 (water) represents the refractive index of water.

The transmitted light spectrum has peaks at wavelengths of 638 nm and 725 nm, with a wavelength of 638 nm being localized at the upper end of the nanohole and a wavelength of 725 nm being localized at the lower end of the nanohole. That is, the LSPR generated in the nanohole shape has a hotspot at the edge portion of the nanohole. The binding of the analyte to the hotspot causes a peak shift of the transmitted (or reflected) light spectrum. By detecting changes in the spectrum, the analyte can be quantified. The peak wavelength of the transmitted light spectrum varies depending on the shape (pitch, hole size, depth, and the like) of the nanohole and the type of material constituting the nanohole. By adjusting these, the nanoholes can be designed to have a peak at a specific wavelength in advance. Thus, it is also possible to quantify the analyte bound to the nanohole structure by irradiating light of a specific single wavelength and measuring the transmittance (or reflectance) thereof. Further, it is also possible to detect a change in the resonance angle by a method of detecting a change in the plasmon resonance angle of the Kretschmann's arrangement type of the SPR detection system which is popular in the past.

The peak shift of the LSPR wavelength caused by binding of the analyte to the hotspot may be detected by Raman scattered light (for example, surface-enhanced Raman scattering spectroscopy) or by fluorescence.

When the analyte is a peptide or a protein, a method for specifically capturing the analyte in the nanostructure as shown in FIG. 1A includes an immunological method using an antibody. Antibodies are proteins and have property of being easily adsorbed on a surface of gold. By using this property, the antibody is adsorbed on the surface of the gold, so that only a target analyte can be specifically captured. Specificity can also be improved by specifically labeling the target analyte with an antibody modified with a labeling substance after capturing the target analyte. A method using the antibody modified with the labeling substance is called a sandwich method. Examples of the labeling substance include a fluorescent substance, an enzyme, a particle, and the like.

When a target analyte is quantified using a nanostructure material in which nanoholes are formed in a general gold thin film shown in FIG. 1A, the antibody is adsorbed to a portion other than the hot spot. Therefore, the analyte is also captured in the portion other than the hotspot. Even if the analyte is captured in the portion other than the hotspot, a spectral shift of the transmitted light does not occur. When the analyte is captured in the portion other than the hotspot, the analyte captured in the hotspot is reduced. As a result, a shift amount of the spectrum of the transmitted light decreases, and detection sensitivity of the analyte decreases. That is, the analyte captured in the portion other than the hotspot becomes background noise, which causes a S/N ratio of a detection signal to deteriorate. Therefore, in order to measure a low-concentration analyte with high sensitivity, it is desirable to reduce an amount of the analyte captured in the portion other than the hot spot.

In the solid-phase carrier of the present embodiment, in the nanostructure in which localized surface plasmon resonance is excited by irradiation of light, a part or all of a region other than a hot spot is covered with a coating layer made of a material that does not excite surface plasmon resonance. The surface of the coating layer has a lower binding affinity for the specific binding substance of the analyte than the surface of the plasmon excitation layer. Therefore, an amount of the specific binding substance that binds to a region other than the hotspot is reduced. As a result, an amount of the analyte captured in the region other than the hotspot can be reduced.

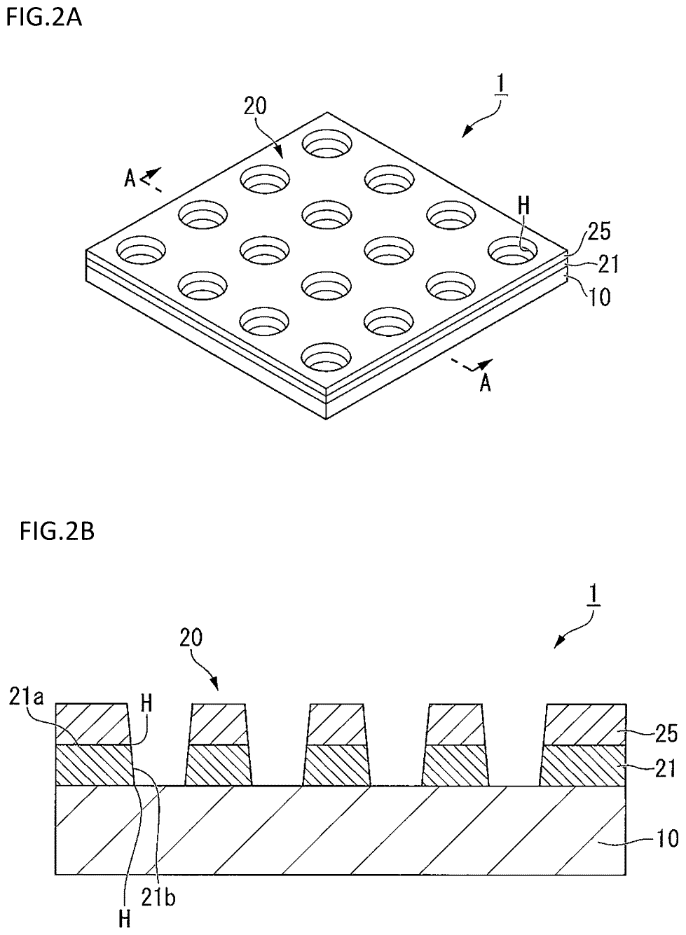

FIG. 2A and FIG. 2B are schematic diagrams showing an example of a solid-phase carrier according to the present embodiment. FIG. 2A is a perspective view of a solid-phase carrier 1 which is an example of a solid-phase carrier. FIG. 2B is a cross-sectional view of the solid-phase carrier 1 shown in FIG. 2A taken along line A-A. The solid-phase carrier 1 shown in FIGS. 2A and 2B includes a light-transmitting substrate 10 and a nanostructured material 20 disposed on one surface of the substrate 10. The nanostructured material 20 includes a plasmon excitation layer 21 and a coating layer 25 that covers a part of the plasmon excitation layer 21.

### <Substrate>

The substrate 10 is not particularly limited, but is preferably a substrate through which a light can transmit. The substrate 10 preferably transmits excitation light used to excite localized surface plasmon resonance. In one embodiment, the substrate 10 is a transparent substrate. Examples of materials of the substrate 10 include glass and a light-transmitting resin. Examples of the light-transmitting resin include, but are not limited to, acrylic resin (such as polymethyl methacrylate), polycarbonate, polyethylene terephthalate, cycloolefin polymer, cycloolefin copolymer, polydimethylsiloxane, polystyrene, and AS resin.

A thickness of the substrate 10 is not particularly limited as long as it can support the nanostructured material 20. The thickness of the substrate 10 is, for example, 0.01 to 10 mm. The thickness of the substrate 10 may be 0.01 to 5 mm, 0.01 to 2 mm, 0.1 to 10 mm, 0.1 to 5 mm, or 0.1 to 2 mm.

### <Nanostructure Material>

The nanostructured material 20 includes a plasmon excitation layer 21 and a coating layer 25.

### (Plasmon Excitation Layer)

The plasmon excitation layer 21 is formed of a material capable of exciting surface plasmon resonance. The plasmon excitation layer 21 has a nanostructure in which localized surface plasmon resonance is excited by irradiation of light.

“A material capable of exciting surface plasmon resonance” refers to, for example, a material having free electrons. The material capable of exciting surface plasmon resonance is, for example, a material having a complex refractive index satisfying a relationship of [real part (n)<extinction coefficient (k)] with respect to incident light.

The complex refractive index is a refractive index of a material having absorption. The complex refractive index (m) can be expressed by the following formula. In the following formula, the real part n represents a normal refractive index, and the imaginary part k is called the extinction coefficient.

m=n−ik

Examples of the material capable of exciting surface plasmon resonance include, for example, a metal. Examples of the metal include, for example, gold, silver, platinum, and aluminum.

“Nanostructure” refers to a three-dimensional structure in which structural units are on the order of nanometers (1 to 1000 nm). The nanostructure may be on the order of nanometers on the longest side of a virtual circumscribed rectangular parallelepiped capable of accommodating the nanostructure.

In the solid-phase carrier 1 shown in FIGS. 2A and 2B, the plasmon excitation layer 21 has a nanohole structure as the nanostructure. The plasmon excitation layer 21 is a layer in which a plurality of nanoholes are formed. The method of disposing the nanoholes is not particularly limited. Examples of the method of disposing the nanoholes include lattice-like disposition.

A pitch P, a diameter D, and a depth T of the nanoholes can be appropriately set according to a wavelength of the irradiation light to be used. The pitch P of the nanoholes is, for example, 50 to 1000 nm. The diameter D of the nanohole is, for example, 10 to 500 nm. The depth T of the nanoholes is, for example, 10 to 500 nm.

The nanostructure is not limited to the nanohole structure, as long as it is a structure in which LSPR is generated by irradiation of light. A shape of the nanohole need not be circular in plan view, and may be, for example, rectangular, oval, or the like in plan view, and is not particularly limited.

A thickness of the plasmon excitation layer 21 is not particularly limited. The thickness of the plasmon excitation layer 21 is, for example, 10 to 500 nm. The thickness of the plasmon excitation layer 21 defines the depth T of the nanohole.

The nanohole structure of the plasmon excitation layer 21 has hotspots H at the upper edge portion and the lower edge portion of the nanohole.

The plasmon excitation layer 21 has a coated region 21a covered by the coating layer 25 and an uncoated region 21b not covered by the coating layer 25. The uncoated region 21b includes a hotspot H. The coated region 21a includes at least a part of a region other than the hotspot H.

Binding affinity to the first specific binding substance 30 of the plasmon excitation layer 21 may be improved by self-assembling monolayer (SAM), chemical modification, or the like. Examples of the chemical modification include modification using a molecule having a functional group at the terminal.

### (Coating Layer)

The coating layer 25 covers a part or all of a region other than the hot spot H of the plasmon excitation layer 21. In the example of FIGS. 2A and 2B, the coating layer 25 covers a region of the plasmon excitation layer 21 where no nanoholes are formed. The hot spots H at the upper and lower edges of the nanoholes of the plasmon excitation layer 21 are not covered by the coating layer 25.

The coating layer 25 is formed of a material that does not excite surface plasmon. “A material that does not excite surface plasmons” is a material that does not have free electrons. As the material that does not excite the surface plasmon, for example, an oxide-based inorganic compound, a resin, or the like can be used. Examples of the oxide-based inorganic compound include a metal oxide. Examples of the metal oxide include, but are not limited to, silicon dioxide (SiO₂), aluminum oxide (Al₂O₃), iron oxide (iron (II) (FeO), iron oxide (III) (Fe₂O₃), triiron tetraoxide (Fe₃O₄), copper oxide (copper (II) oxide (CuO), copper (I) oxide (Cu₂O)), titanium oxide (TiO₂), and the like. Examples of the resin include, but are not limited to, silicone resins, acrylic resins, polycarbonates, polyethylene terephthalate, polydimethylsiloxane, and fluorine-based resins. The material that does not excite surface plasmon resonance may be a dielectric, and the coating layer 25 may be a dielectric layer.

The surface of the coating layer is formed to have a lower binding affinity for the specific binding substance of the analyte than the surface of the plasmon excitation layer 21. Therefore, it is preferable that the coating layer is formed of a material having low binding affinity or adsorptivity for a specific binding substance.

When the specific binding substance is a protein such as an antibody, the coating layer is preferably formed of a material having low adsorptivity or binding affinity for the protein. For example, a material in which the surface of the coating layer becomes hydrophilic can be used. For example, the surface of the SiO₂, on which OH groups are highly exposed, has hydrophilic properties. Therefore, it is difficult for proteins to adsorb. The surface of the coating layer may be modified to have a low binding affinity or adsorptivity for a specific binding substance. For example, the surface of the coating layer may be hydrophilized. For example, chemical modification by a hydrophilic molecule such as polyethylene glycol may be performed.

A thickness of the coating layer 25 is not particularly limited. The thickness of the coating layer 25 affects the peak wavelength of the transmitted light spectrum. Therefore, the thickness of the coating layer 25 may be appropriately set in accordance with the wavelength of the irradiation light to be used. The thickness of the coating layer 25 is, for example, 1 to 500 nm.

### <Method for Producing Solid-Phase Carrier>

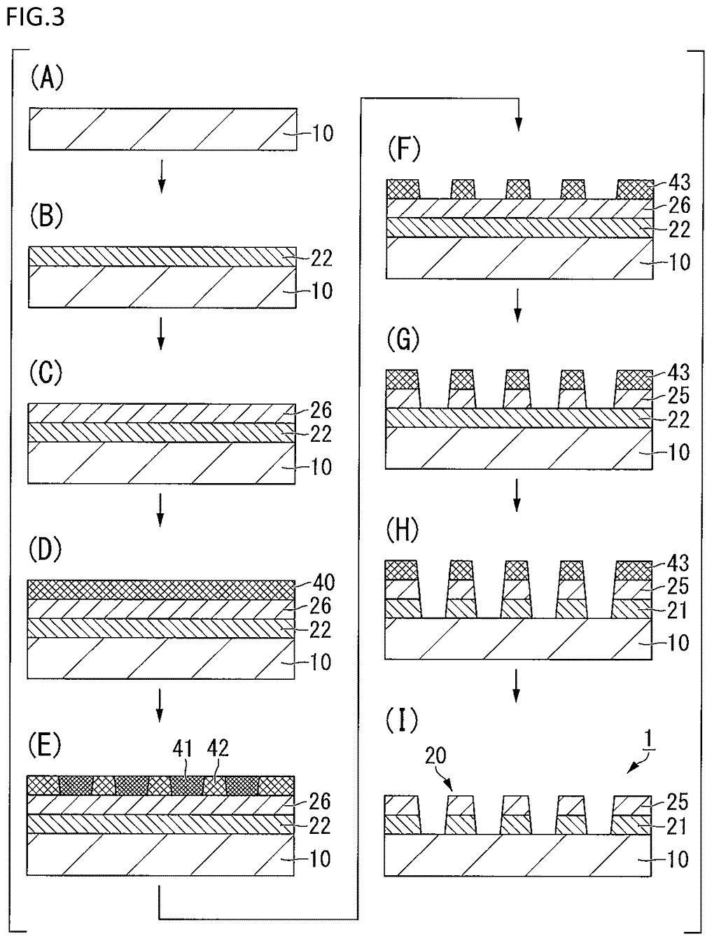

FIG. 3 is a schematic diagram illustrating an example of a method for producing a solid-phase carrier.

A suitable substrate such as a glass substrate is polished and cleaned to prepare a substrate 10 having a smooth surface (FIG. 3A).

Next, a layer 22 of a material capable of exciting surface plasmon resonance is formed on the substrate 10 (FIG. 3B). The layer 22 can be formed by a known method. Examples thereof include a vacuum film forming method (a sputtering method, a vacuum vapor deposition method, and the like). When nanoholes are formed as nanostructures, the thickness of the layer 22 may be equal to or greater than the depth of the nanoholes finally formed. The thickness of the layer 22 is preferably the same as the depth of the finally formed nanohole.

Next, a layer 26 of a material that does not excite surface plasmon resonance is formed on the layer 22 (FIG. 3C). The layer 26 can be formed by a known method. Examples thereof include a vacuum film forming method and a spin coating method. For example, when an oxide-based inorganic compound is used as a material that does not excite surface plasmon resonance, a vacuum film formation method can be used. When a resin is used as a material that does not excite surface plasmon resonance, a spin coating method can be used. In a case where a metal oxide is used as a resist in a step described later, since the resist film is formed by a vacuum film forming method, it is preferable that the layer 22, the layer 26, and the resist film be continuously formed by the vacuum film forming method. In a case of forming the nanostructure by a laser drawing method, the layer 26 is preferably formed of a material having a low thermal conductivity. Such materials include, for example, SiO₂.

A thickness of the layer 26 may be equal to or greater than the thickness of the coating layer 25 to be finally formed.

Next, a resist film 40 is formed on the layer 26. The resist film 40 can be formed by a known method using a known resist composition. Examples thereof include a vacuum film forming method and a spin coating method.

When an electron beam lithography method is used for forming the nanostructure, for example, a positive resist (ZEP-520 or the like as a typical resist material) can be used. When the laser drawing method is used for forming the nanostructure, it cannot be used in general g-line resist and i-line resist because the resolution limit is lower. When the thermal energy of the Gaussian distribution of the focusing laser is used, a metal resist (an oxide of a transition metal or the like as a typical resist material) can be used.

A thickness of the resist film 40 is not particularly limited, and may be any thickness that can be used as a mask in a process described later. The thickness of the resist film 40 is, for example, about 50 to 1000 nm.

In order to improve an adhesion of the resist film 40 to the layer 26, an adhesion layer may be formed between the layer 26 and the resist film 40. Examples of the adhesion layer include an inorganic thin film made of chromium, germanium, titanium, or the like, and an organic thin film made of a silane coupling agent or the like. When the layer 22, the layer 26, and the resist film 40 are formed by a vacuum film forming method, it is possible to form a continuous film, and therefore it is preferable to use an inorganic thin film as the adhesion layer. When an alkali developer is used in the developing step, chromium that is not dissolved in the alkali developer may be used.

A thickness of the adhesion layer is, for example, about 1 to 5 nm, preferably about 1 to 2 nm.

Next, a nanostructured pattern (e.g., nanohole pattern) is exposed by an electron beam drawing method, a laser drawing method, or the like (FIG. 3E). A exposed portion 41 and an unexposed portion 42 are formed on the resist film 40 by exposure. In the laser drawing method, since it is possible to perform drawing at a higher speed than in the electron beam drawing method, it is possible to produce the nanostructure in a large area in a short time. Therefore, it is possible to provide an inexpensive solid-phase carrier having high mass productivity.

Next, development is performed using a developer to form a resist pattern 43 (FIG. 3F).

Next, the layer 22 and the layer 26 are etched using the resist pattern 43 as a mask (FIG. 3G and FIG. 3H). Etching can be performed by a known method. For example, a dry etching method such as reactive ion etching (RIE) or ion milling can be used. Examples of the dry etching include etching using an Ar gas. When forming the nanoholes, the layer 22 is etched so as to form the nanoholes having a desired depth. When forming the nanoholes, the layer 22 of the nanohole forming portion is preferably entirely removed by etching.

Next, the resist pattern 43 is removed (FIG. 3I). This makes it possible to obtain the solid-phase carrier 1 in which the nanostructured material 20 having a desired nanostructure is disposed on the substrate 10. The resist pattern 43 can be removed by a known method. For example, an immersion method using an alkali or an organic solvent, a dry etching method, or the like can be used.

<Solid-Phase Carrier Bound with First Specific Binding Substance>

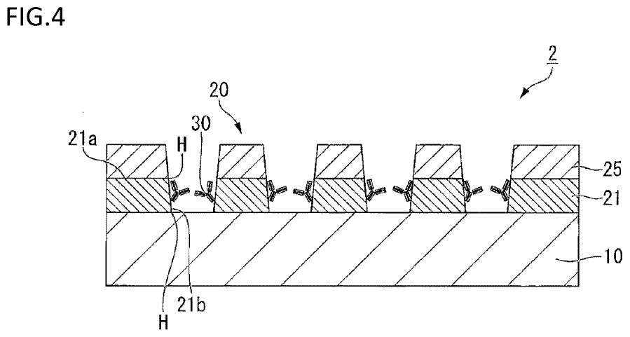

The solid-phase carrier may be a carrier on which a first specific binding substance of an analyte is bound to an uncoated region of the plasmon excitation layer (see FIG. 4).

In the solid-phase carrier 2 shown in FIG. 4, the first specific binding substance 30 of the analyte is bound to the uncoated region 21b.

The binding method of the first specific binding substance 30 to the uncoated region 21b can be performed using a known method. When the plasmon excitation layer 21 is formed of a metal such as gold, the metal surface generally has a property of easily adsorbing an organic substance. On the other hand, the surface of the coating layer 25 has a lower binding affinity for the first specific binding substance 30 than the uncoated region 21b.

Therefore, for example, a solution containing the first specific binding substance 30 is supplied to the surface of the solid-phase carrier 1 on which the nanostructured material 20 is disposed. Accordingly, the first specific binding substance 30 can be adsorbed onto the surface of the uncoated region 21b. On the other hand, the first specific binding substance 30 hardly adsorbs on the surface of the coating layer 25. As a result, the solid-phase carrier 2 in which the first specific binding substance 30 is bound only to the uncoated region 21b can be obtained.

In the binding reaction of the first specific binding substance 30, a surfactant or the like may be added to the solution containing the first specific binding substance 30 to further suppress the first specific binding substance 30 to the coating layer 25.

A supply of the solution containing the first specific binding substance 30 to the surface of the solid-phase carrier 1 on which the nanostructured material 20 is disposed can be performed by attaching a well-shaped well to the surface and injecting the solution containing the first specific binding substance 30 therein. The Well-shaped well can be made of, for example, a resin such as dimethylsiloxane (PDMS). A binding reaction of the first specific binding substance 30 to the uncoated region 21b may be carried out at room temperature (20 to 30° C.) or may be carried out in a thermostat maintained at a predetermined temperature. A temperature of the binding reaction can be appropriately selected according to a type of the first specific binding substance 30.

After the binding reaction of the first specific binding substance 30, the solid-phase carrier 2 may be washed. The washing process can be performed by repeating a process of supplying washing solution to the surface of the solid-phase carrier 2 and removing the washing solution about 1 to 3 times. Examples of the washing solution include a solution obtained by adding a nonionic surfactant such as Tween 20 to a buffer solution such as PBS. By performing the washing process, unreacted first specific binding substances 30 can be removed.

### <Method (1) for Measuring Analyte>

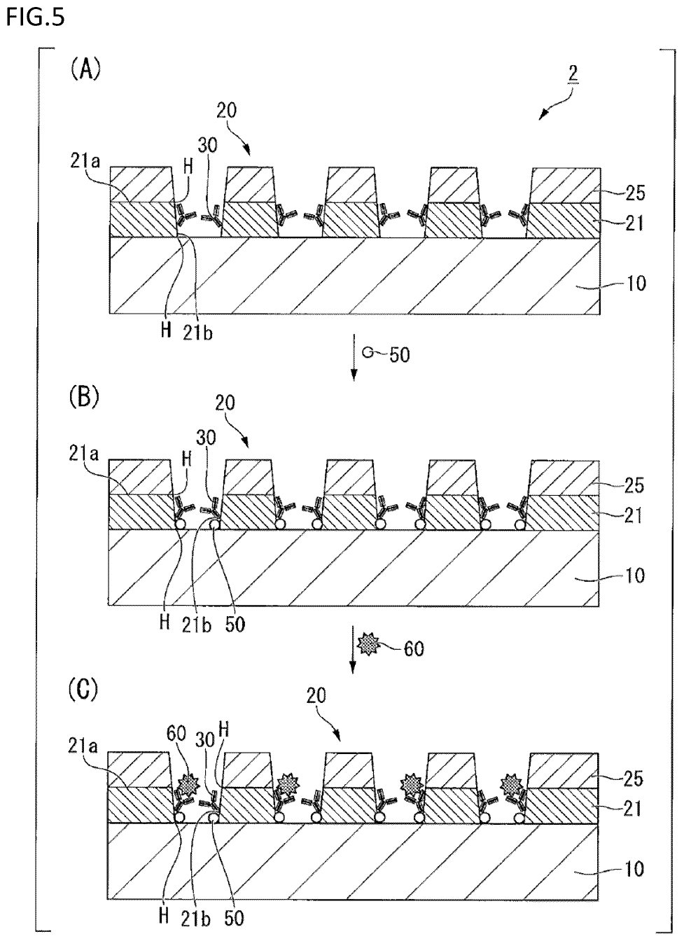

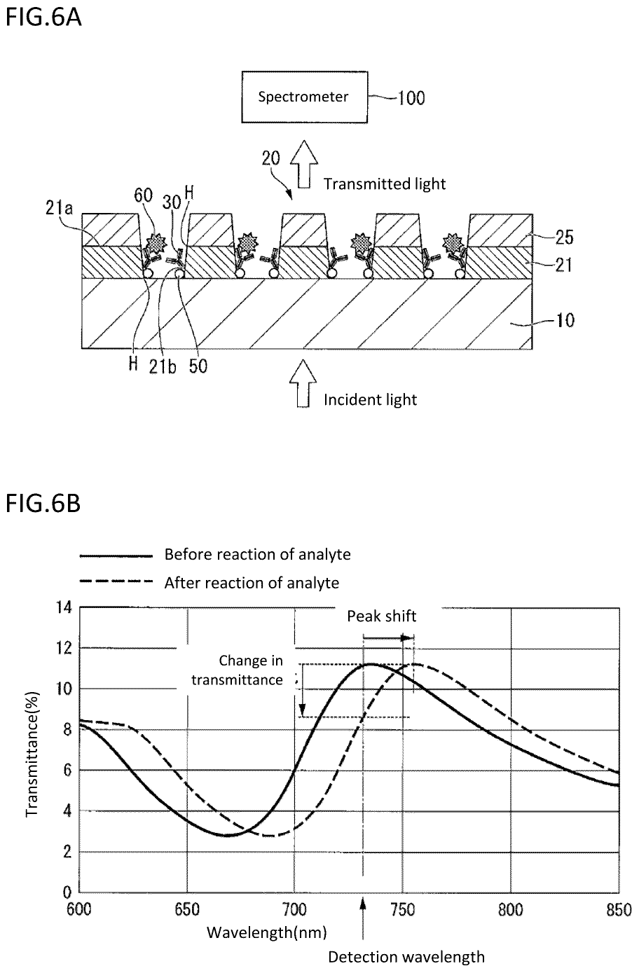

FIG. 5 and FIG. 6A are schematic diagrams illustrating a method of measuring an analyte using the solid-phase carrier 2.

The measurement method can include a step of blocking a surface of the solid-phase carrier 2 on which the nanostructured material 20 is disposed (blocking step; FIG. 5B), a step of binding the analyte to the uncoated region 21b via binding to the first specific binding substance 30 (analyte capturing step; FIG. 5C), and a step of detecting LSPR (LSPR detection step; FIG. 6A).

### (Blocking Step)

A surface of the solid-phase carrier 2 to which the first specific binding substance 30 is not bound can be blocked by performing the blocking process. This makes it possible to reduce non-specific adsorption on the solid-phase carrier 2.

The blocking can be performed by supplying a solution containing a blocking agent 50 to the surface of the solid-phase carrier 2 on which the nanostructured material 20 is disposed.

As the blocking agent, a known blocking agent can be used without any particular limitation. Examples of the blocking agent include biologically-derived protein-based blocking agents commonly used in the field of biochemistry such as bovine serum albumin (BSA), casein, skim milk, and fish gelatin; and synthetic polymer compounds such as polyethylene glycol (PEG) and polyvinyl alcohol (PVA). One kind of the blocking agent may be used alone, or two or more kinds there of may be used in combination. For example, PEG and a protein-based blocking agent may be used in combination.

Examples of a buffer for the blocking solution include, but are not limited to, a buffer solution such as phosphate buffer, PBS (Phosphate-buffered saline), Tris buffer, or HEPES buffer, or a buffer solution obtained by adding a surfactant such as Tween 20 to the buffer solution as described above.

A reaction temperature of the blocking reaction is, for example, 20 to 40° C. A reaction time of the blocking reaction is, for example, 10 to 120 minutes.

After the blocking reaction, the solid-phase carrier 2 may be washed. Unreacted blocking agents can be removed by performing the washing process.

### (Analyte Capture Step)

An analyte 60 is bound to the first specific binding substance 30 by supplying a solution containing the analyte 60 to the surface on which the nanostructured material 20 of the solid-phase carrier 2 is disposed. As a result, the analyte 60 can be captured by the solid-phase carrier 2.

A solution containing an analyte may be a biological sample. The biological sample may be appropriately diluted with a dilution buffer. As the dilution buffer, for example, a buffer solution (PBS or the like) or the like can be used. The dilution buffer may also include a blocking agent and/or a surfactant (such as Tween 20) to reduce non-specific adsorption.

A condition for the binding reaction between the first specific binding substance and the analyte can be appropriately selected according to types of the analyte and the first specific binding substance. For example, when the analyte is a protein or a peptide and the first specific binding substance is an antibody, the reaction temperature may be 20° C. or higher, 30° C. or higher, 35° C. or higher, and the like. An upper limit of the reaction temperature is not particularly limited, and examples thereof include 50° C. or lower, 45° C. or lower, and 40° C. or lower, and the like. Examples of the reaction temperature range include 20 to 50° C., 30 to 45° C., and 30 to 40° C., and the like.

A reaction time is not particularly limited, and may be a time sufficient for the binding reaction between the first specific binding substance and the analyte to proceed. The reaction time can be appropriately selected according to types of the analyte and the first specific binding substance. When the analyte is a protein or a peptide and the first specific target substance is an antibody, the reaction time includes, for example, 3 to 200 minutes. A lower limit of the reaction time is not particularly limited, and examples thereof include 3 minutes or more, 5 minutes or more, 10 minutes or more, 20 minutes or more, 30 minutes or more, and the like. An upper limit of the reaction time is not particularly limited, and examples thereof include 200 minutes or less, 180 minutes or less, 120 minutes or less, and 100 minutes or less, and the like. Examples of the reaction time range include 5 to 180 minutes, 10 to 120 minutes, 20 to 100 minutes, and 30 to 10 minutes, and the like.

After the binding reaction of the analyte, the solid-phase carrier 2 may be washed. By performing the washing process, unreacted target substances and contaminants can be removed.

The blocking step and the analyte capturing step may be performed by forming a structure in which a reaction solution can be placed, on a surface of the solid-phase carrier 2 on which the nanostructured material 20 is disposed. For example, a blocking reaction or an analyte binding reaction can be performed by placing the reaction solution in the structure in which the reaction solution can be placed for a predetermined period of time. Examples of the structure in which the reaction solution can be placed include a well structure, a channel structure, and the like. The well structure can be formed, for example, by laminating a resin sheet or the like having a through-hole having a desired size on a surface on which the nanostructured material 20 of the solid-phase carrier 2 is disposed. The channel structure can be formed, for example, by disposing a resin sheet on which a channel is formed, and sealing an upper portion of the channel with another resin sheet. The material of the resin sheet is not particularly limited, and examples thereof include PDMS, a cycloolefin polymer, and a cycloolefin copolymer.

### (LSPR Detection Step)

Light is irradiated to the solid-phase carrier 2 to which the analyte is not bound (FIG. 5B) and to the solid-phase carrier 2 to which the analyte is bound (FIG. 5C), as shown in FIG. 6A, from a surface on the substrate 10 side. The light transmitted to a surface where the nanostructured material 20 is disposed is detected by a spectrometer 100. A measurement of the transmitted light may be performed by a spectroscopically detectable instrument. The spectrometer 100 detects a transmitted light spectrum having a peak at a specific wavelength.

In the solid-phase carrier 2 illustrated in FIG. 5C, since the analyte 60 is captured, a transmitted light spectrum, in which a peak wavelength is shifted from that of a transmitted light spectrum of the solid-phase carrier 2 illustrated in FIG. 5B, is detected. From an amount of this peak shift, an amount of the analyte 60 captured by the solid-phase carrier 2 can be detected.

FIG. 6B shows an example of a spectral shift of transmitted light.

The peak shift of the transmitted light spectrum by LSPR means that a transmittance at a specific detection wavelength changes (see FIG. 6B). Therefore, detection of LSPR may be performed using light of a single wavelength. A wavelength used can be, for example, near a peak wavelength of the transmitted light spectrum in the solid-phase carrier 2 before binding the analyte. The analyte can be quantified by detecting a change in transmittance at a specific wavelength with respect to the solid-phase carrier 2 to which the analyte is not bound (FIG. 5B) and the solid-phase carrier 2 to which the analyte is bound (FIG. 5C). Since it is not necessary to use a high-cost spectrometer, there is an advantage that a cost reduction of the detection device can be expected.

As shown in FIGS. 1A and 1B, when LSPR in gold nanoholes having a pitch P of 400 nm, a diameter D of 150 nm, and a depth T of 100 nm is calculated by an electromagnetic wave simulation, a transmitted light spectrum having peaks at 638 nm and 725 nm is obtained. When the analyte is adsorbed on the hotspot of the nanohole, the peaks shift toward long wavelength side. The peak shift amount is proportional to the adsorption amount of the analyte, and is evaluated, in units of nm/RIU (Refractive Index Unit) as the peak shift amount per unit refractive index change amount of the analyte, as a value indicating a sensitivity as a device. As a sensitivity of LSPR using nanostructure such as gold nanohole, there is a report example of 100˜1000 nm/RIU. This peak shift occurs due to the analyte bound to the hotspot. Analytes adsorbed other than the hotspot cannot be detected as a change in the LSPR mode, and are detected as a change in the normal SPR mode. Therefore, in detection of LSPR, they are a factor that lowers detection accuracy as background noise.

With respect to the solid-phase carrier 1 in a state in which a solution of the first specific binding substance 30 and the analyte is injected into a nanostructured surface, an amount of a substance bound to the hotspot can be measured in real time as long as light incidence and transmitted light detection can be maintained. For example, by forming a channel structure in which a solution can be placed on the nanostructured surface, it is possible to inject the solution into the nanostructured surface while maintaining the light incidence and the transmitted light detection.

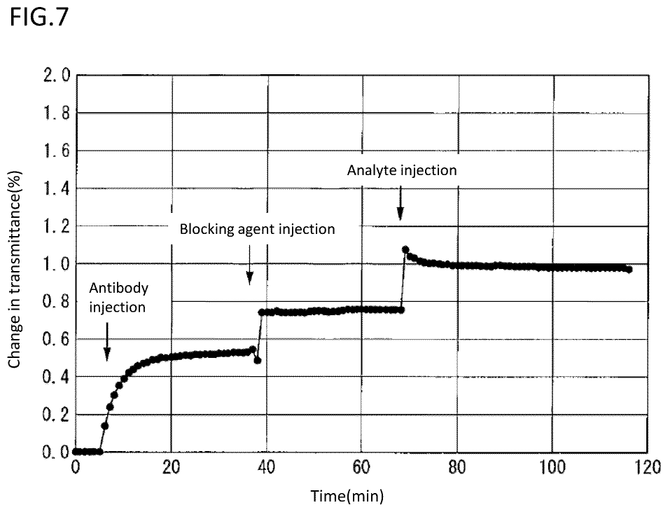

FIG. 7 shows an example in which a change in transmittance at a specific wavelength (see FIG. 6B), when the reaction of the first specific binding substance 30, the blocking step, and the analyte capturing step are performed on the solid-phase carrier 1, is measured in real time.

### <Method (2) of Measuring Analyte>

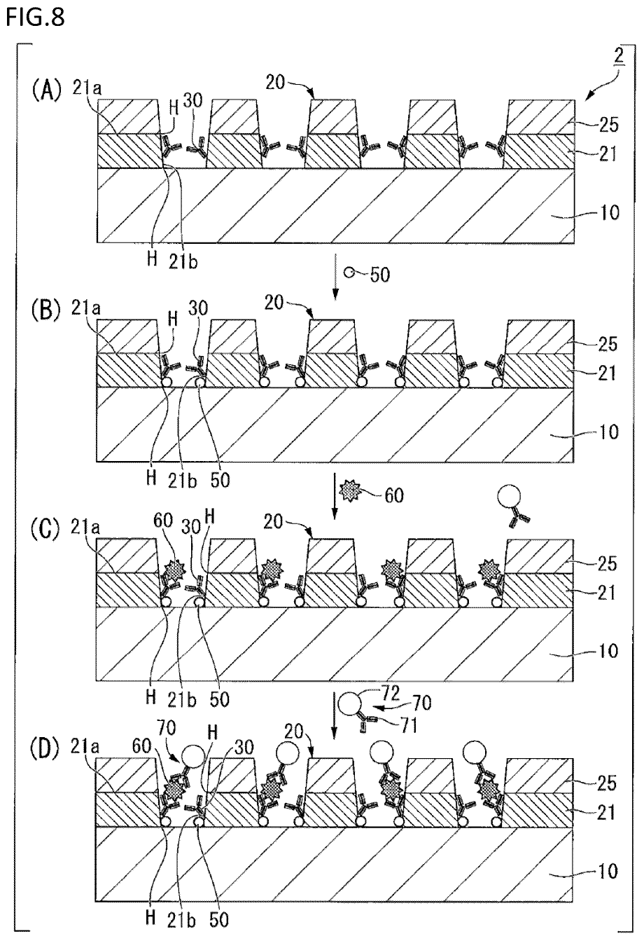

The method for measuring an analyte may include a step of binding a labeling substance to the analyte (labeling substance binding step; FIG. 8D) between the analyte capturing step and the LSPR detection step. FIG. 8 is a schematic diagram illustrating a method of measuring an analyte including the labeling substance binding step.

The blocking step (FIG. 8B) and the analyte capturing step (FIG. 8C) can be performed in the same manner as described above.

### (Labeling Substance Binding Step)

A labeling conjugate 70 include a second specific binding substance 71 to which a labeling substance 72 is bound.

The second specific binding substance 71 is appropriately selected according to the analyte. The second specific binding substance 71 may be the same as or different from the first specific binding substance 30. From the viewpoint of forming a complex of the first specific binding substance, the analyte, and the second specific binding substance, the second specific binding substance is preferably different from the first specific binding substance. For example, when the analyte is a peptide or a protein, the first specific binding substance and the second specific binding substance may be antibodies that bind to epitopes different from each other. For example, when the analyte is a nucleic acid such as RNA or DNA, the first specific binding substance and the second specific binding substance may be nucleic acid probes (polynucleotide probes or oligonucleotide probes) that hybridize to regions different from each other.

Examples of the labeling substance 72 include nanoparticles, enzymes, and fluorescent substances. Nanoparticles are particles on the order of nanometers (1-1000 nm). Examples of particle size of the nanoparticles include 10 nm to 300 nm. The material of the nanoparticle is not particularly limited. Examples of the nanoparticles include resin beads (polystyrene, glycidyl methacrylate, and the like), magnetic beads, and metal nanoparticles. Examples of enzyme include peroxidase and alkaline phosphatase. Examples of fluorescent substance include carboxyfluorescein (FAM), 6-carboxy-4′,5′-dichloro2′,7′-dimethoxyfluorescein (JOE), fluorescein isothiocyanate (FITC), tetrachlorofluorescein (TET), 5′-hexachloro-fluorescein-CE phosphoramidite (HEX), Cy3, Cy5, Alexa 568, and Alexa647.

As the labeling substance 72, a substance, which makes a peak shift of transmitted light spectrum when the labeling conjugate 70 is bound to the analyte 60 larger than a peak shift of transmitted light spectrum when only the analyte 60 is captured, is preferably used. The labeling substance 72 is preferably, for example, a nanoparticle. A type of the nanoparticle may be selected such that a peak shift amount of transmitted light spectrum increases due to interaction between the nanoparticle and the nanostructure included in the plasmon excitation layer 21.

Binding of the labeling substance 72 to the second specific binding substance 71 can be performed by a known method. Examples thereof include a method utilizing physical adsorption, a method of modifying the labeling substance by a functional group that reacts with a functional group possessed by the second specific binding substance (for example, amino group modification, hydroxyl group modification, carboxy group modification, succinimide group modification, and the like), and a method of utilizing biotin-avidin binding.

Binding reaction of the labeling conjugate 70 can be performed by supplying a solution containing the labeling conjugate 70 to a surface of the solid-phase carrier 2 on which the nanostructured material 20 is disposed.

A condition for binding reaction of the labeling substance can be appropriately selected according to types of the analyte and the second specific binding substance. For example, when the analyte is a protein or a peptide and the second specific binding substance is an antibody, a reaction temperature may be 20° C. or higher, 30° C. or higher, 35° C. or higher, or the like. An upper limit of the reaction temperature is not particularly limited, and examples thereof include 50° C. or lower, 45° C. or lower, and 40° C. or lower. Examples of the reaction temperature range include 20 to 50° C., 30 to 45° C., and 30 to 40° C.

Reaction time is not particularly limited, and may be a time sufficient for the binding reaction between the second specific binding substance and the analyte to proceed. The reaction time can be appropriately selected according to types of the analyte and the second specific binding substance. When the analyte is a protein or a peptide and the second specific target substance is an antibody, the reaction time includes, for example, 3 to 200 minutes. A lower limit of the reaction time is not particularly limited, and examples thereof include 3 minutes or more, 5 minutes or more, 10 minutes or more, 20 minutes or more, 30 minutes or more, and the like. An upper limit of the reaction time is not particularly limited, and examples thereof include 200 minutes or less, 180 minutes or less, 120 minutes or less, and 100 minutes or less, and the like. Examples of the reaction time range include 5 to 180 minutes, 10 to 120 minutes, 20 to 100 minutes, and 30 to 10 minutes, and the like.

After the binding reaction of the labeling conjugate 70, the solid-phase carrier 2 may be washed. By performing the washing process, unreacted labeling conjugate 70 can be removed.

By binding the labeling conjugate 70 to the analyte 60, an amount of peak shift of the transmitted light spectrum due to LSPR can be increased. Therefore, detection sensitivity of the analyte can be improved. In addition, specificity for the analyte can be improved.

The labeling substance binding step may be performed by forming a structure in which a reaction solution can be placed on a surface of the solid-phase carrier 2 on which the nanostructured material 20 is disposed, similar to the blocking step and the analyte capturing step.

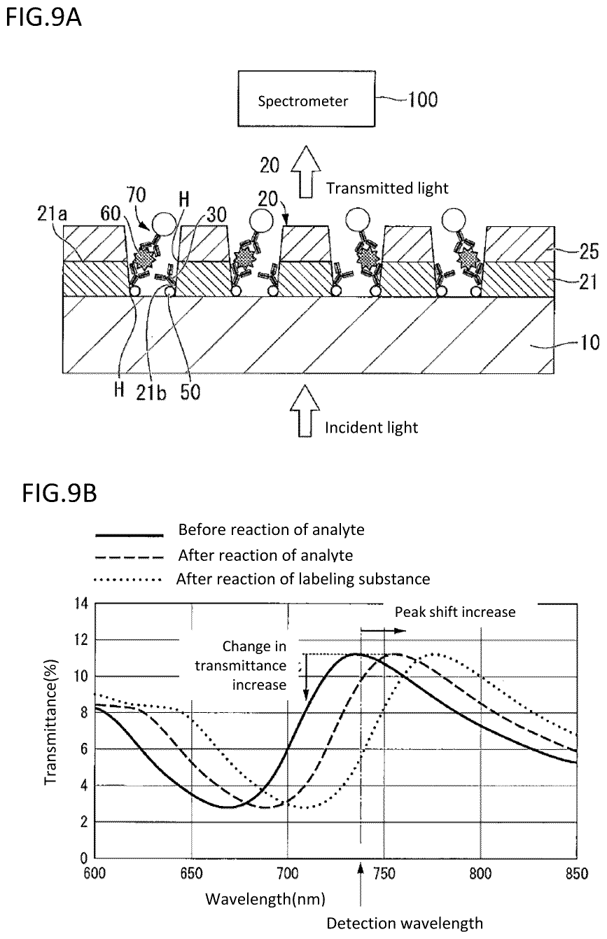

The LSPR detection step (FIG. 9A) can be performed in the same manner as described above, except that a solid-phase carrier (FIG. 8D) to which the labeling conjugate 70 is bound is used.

FIG. 9B shows an example of a spectral shift of transmitted light.

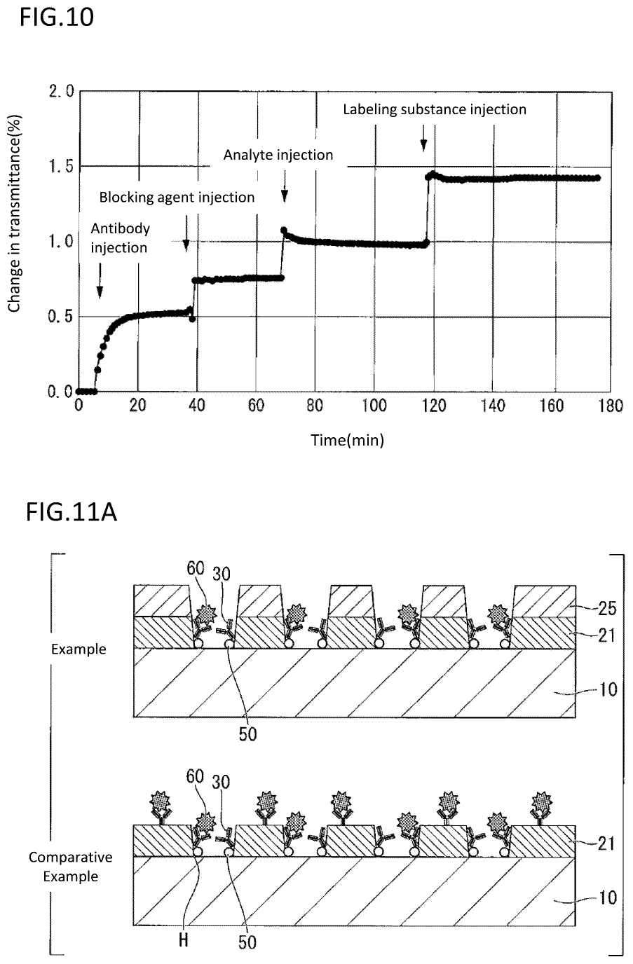

FIG. 10 shows an example in which a change in transmittance at a specific wavelength (see FIG. 9B) is measured in real time through conducting the reaction of the first specific binding substance 30, the blocking step, the analyte capturing step, and the labeling substance binding step on the solid-phase carrier 1.

In the solid-phase carrier of the present embodiment, a part or all of a region other than the hot spot of the plasmon excitation layer is covered with the coating layer. The coating layer has a lower binding affinity for the first specific binding substance than the plasmon excitation layer. Therefore, when the first specific binding substance is bound to the solid-phase carrier, binding of the first specific binding substance to the region other than the hotspot is reduced. As a result, S/N ratio can be improved, and detection sensitivity of the analyte can be improved.

### [Kit for Measuring Analyte]

A second embodiment of the present invention is a kit for measuring analyte. The kit includes the solid-phase carrier of the first embodiment and the second specific binding substance, of the analyte, to which the labeling substance is bound.

The solid-phase carrier is the same as the solid-phase carrier of the first embodiment. The solid-phase carrier may be one in which a first specific binding substance is bound to an uncoated region of the plasmon excitation layer, or one in which a first specific binding substance is not bound.

The second specific binding substance to which the labeling substance is bound is the same as that described for the labeling substance. The labeling substance is preferably a nanoparticle.

### (Optional Configuration)

The measurement kit of the present embodiment may include any configuration in addition to the above-described configuration. Examples of the optional configuration include various reagents such as a reagent for processing a sample (for example, a diluent), a washing solution, a blocking solution, and a buffer solution, standard reagents, and instructions for use.

The measurement kit of the present embodiment may include a standard reagent. The standard reagent is a reagent for preparing a calibration curve of an analyte to be detected. As the standard reagent, for example, a purified product of the same kind as the analyte can be used.

Since the measuring kit of the present embodiment includes the solid-phase carrier and the labeling substance according to the above embodiments, a low-concentration analyte can be measured with high sensitivity.

### Examples

Hereinafter, the present invention will be described by way of Examples, but the present invention is not limited to the following Examples.

### Examples

### (Preparation of Solid-Phase Carrier)

A solid-phase carrier was prepared according to the method shown in FIG. 3. As a material capable of exciting the surface plasmon resonance, gold was used for forming the plasmon excitation layer 21. As a material that does not excite surface plasmon resonance, SiO₂ was used for forming the coating layers 25.

Specifically, a 2 nm chromium layer (adhesive layer), a 50 nm gold thin film layer, a 50 nm SiO₂ coating layer, and a 50 nm thermal resist layer (a film formed from tungsten by reactive sputtering using a mixed gas of oxygen and argon) were formed on a polished and cleaned BK7 glass by sputtering. Each layer was continuously formed in the same sputtering apparatus to eliminate an effect of surface oxidation. Next, a semiconductor laser having a wavelength of 405 nm was focused on the resist surface with an objective lens, and a hole pattern (diameter: 150 nm) was created at a pitch of 400 nm. It was then developed with 2.38% tetramethylammonium hydroxide (TMAH) developer for 20 minutes. As a result, a hole resist pattern with a pitch of 400 nm was prepared.

Then, the SiO₂ layer as the coating layer was etched using CHF₃ gases by a reactive ion-etching (RIE) method. Then, the gold thin film layer was etched by Ar gas. A hot resist layer was then removed by immersion in 2.38% tetramethylammonium hydroxide (TMAH) developer overnight. Thus, a solid-phase carrier A, in which nanohole structure (pitch: 400 nm, diameter: 150 nm, and depth: 100 nm) was disposed on a glass surface, was fabricated.

### (Anti-PSA Antibody Binding)

A plurality of well-shaped wells each having a diameter of 6 mm and a height of 10 mm, which were prepared by PDMS, were attached to a surface of the solid-phase carrier on which the nanostructures were disposed. To each well-shaped well, 100 μL of a solution of anti-PSA antibody (Model No. 4P33-1H12, manufactured by HyTest Co., Ltd.) prepared at 5 μg/mL in PBS was injected and incubated at 37° C. for 30 minutes. Thereafter, washing was performed three times with PBST.

### (Blocking Step)

In order to suppress non-specific adsorption on the solid-phase carrier A, the surface on which the nanostructure of the solid-phase carrier A was disposed was blocked with BSA (bovine serum albumin, model number 37525, manufactured by Thermo Fisher). After the blocking, it was washed 3 times with PBST.

### (Analyte Capturing Step)

PSA was used as the analyte. PSA antigen (Model No. 8P78, manufactured by HyTest Co., Ltd.) was diluted with PBST to prepare a dilution series of PSA solutions of 0 to 1 ng/ml. 100 μL of the dilution series of PSA solution was injected into each well and allowed to react at 37° C. for 60 minutes. After the binding reaction, the mixture was washed three times with PBST.

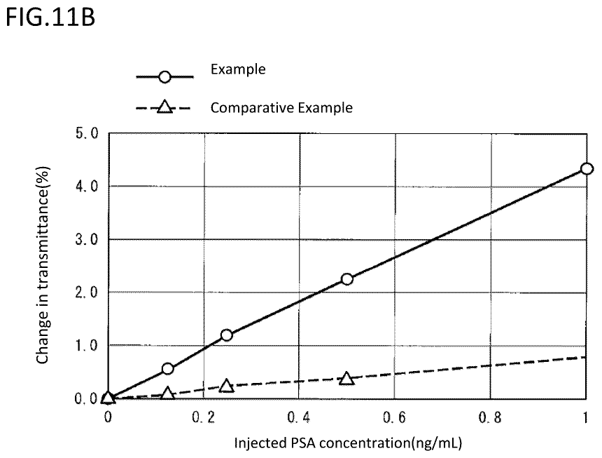

A schematic diagram of the solid-phase carrier A after the analyte capturing step in the example is shown in FIG. 11A (upper diagram).

### (LSPR Detection Step)

White light was irradiated from a glass substrate side, and transmittance of transmitted light having a wavelength of 725 nm in each reaction region was measured by a spectrometer. An amount of change in transmittance at each PSA concentration was plotted graphically with setting transmittance at a PSA concentration of 0 ng/ml as a standard.

### Comparative Example

### (Preparation of Solid-Phase Carrier)

A solid-phase carrier was prepared according to the method shown in FIG. 3, except that layer 26 was not formed.

Specifically, a 2 nm chromium layer (adhesive layer), a 50 nm gold thin film layer, and a 50 nm thermal resist layer (a film formed from tungsten by reactive sputtering using a mixed gas of oxygen and argon) were formed on a surface of a polished and cleaned BK7 glass by sputtering. A hole resist pattern having a pitch of 400 nm was prepared in the same manner as in Example 1.

Next, the gold thin film layer was etched by Ar gas by a reactive ion etching (RIE) method. The hot resist layer was then removed by immersion in 2.38% tetramethylammonium hydroxide (TMAH) developer overnight. Thus, a solid-phase carrier B in which nanohole structure (pitch: 400 nm, diameter: 150 nm, depth: 50 nm) was disposed on the glass surface was prepared.

### (Binding of Anti-PSA Antibody, Analyte Capturing Step)

The binding of the anti-PSA antibody and the capture of the analyte were performed in the same manner as in the example except that the solid-phase carrier B was used instead of the solid-phase carrier A.

A schematic diagram of the solid-phase carrier B after the analyte capturing step in the comparative example is shown in FIG. 11A (lower diagram).

### (LSPR Detection Step)

White light was irradiated from the glass substrate side, and transmittance of transmitted light having a wavelength of 725 nm in each reaction region was measured by a spectroscope. An amount of change in transmittance at each PSA concentration was plotted graphically with setting transmittance at a PSA concentration of 0 ng/ml as a standard.

A graph plotting a change in transmittance is shown in FIG. 11B. In each PSA concentration, the transmittance change amount was larger in the Example than in the Comparative Example. The amount of change in transmittance is affected by an amount of an analyte captured on a portion other than the hotspot. In the comparative example, since the portion other than the hotspot is not covered with the SiO₂ layers, the analyte is also captured in the region other than the hotspot. As a result, it is considered that an amount of the analyte captured on the hotspot decreased and the amount of change in transmittance decreased. Therefore, an amount of change in transmittance when measuring a low-concentration analyte becomes small, and it becomes difficult to measure the analyte.

On the other hand, in the examples, it was confirmed that a change in transmittance can be sufficiently detected even at a PSA concentration of 0.1 ng/ml. These results show that the analyte can be detected with high sensitivity in the example.

From the above results, it was confirmed that the analyte can be detected with high sensitivity by using a solid-phase carrier in which a region other than a hot spot is coated with a coating layer.

### INDUSTRIAL APPLICABILITY

According to the present invention, a solid-phase carrier and a kit for measuring an analyte, which can reduce an amount of the analyte captured on a region other than a hotspot, are provided.

While preferred embodiments of the present invention have been described above, the present invention is not limited to these embodiments. Additions, omissions, substitutions, and other changes may be made without departing from the spirit of the present invention. The invention is not limited by the foregoing description, but only by the scope of the appended claims.

What is claimed is:

1. A solid-phase carrier used for measuring an analyte, comprising:
a substrate; and
a nanostructured material disposed on one surface of the substrate,
wherein the nanostructured material comprises:
a plasmon excitation layer formed of a material capable of exciting surface plasmon resonance and having a nanostructure in which localized surface plasmon resonance is excited by irradiation of light; and
a coating layer formed of a material that does not excite surface plasmon resonance,
wherein the plasmon excitation layer includes a coated region covered by the coating layer and an uncoated region not covered by the coating layer,
wherein the uncoated region includes a hotspot where localized surface plasmon resonance occurs, and
wherein a surface of the coating layer has a lower binding affinity for a specific binding substance of the analyte than a surface of the plasmon excitation layer.

2. The solid-phase carrier according to claim 1, wherein a first specific binding substance is bound to the uncoated region, the first specific binding substance being capable of binding to the analyte.

3. The solid-phase carrier according to claim 1, wherein the substrate is a light-transmitting substrate.

4. A kit for measuring an analyte, comprising:
the solid-phase carrier according to claim 1; and
a second specific binding substance labeled with a labeling substance, the second specific binding substance being capable of binding to the analyte.
